(19) United States
(12) Patent Application Publication
(10) Pub. No.: US 2021/0002205 A1
(43) Pub. Date: Jan. 7, 2021

(54) PROCESS FOR THE SEPARATION OF ALKANOLAMINE FROM A MIXTURE COMPRISING AN ALIPHATIC PRIMARY DIAMINE

(71) Applicant: BASF SE, Ludwigshafen am Rhein (DE)

(72) Inventor: Fabien RIVOLLET, Caluire et Cuire (FR)

(73) Assignee: BASF SE, Ludwigshafen am Rhein (DE)

(21) Appl. No.: 16/975,878

(22) PCT Filed: Feb. 27, 2019
(86) PCT No.: PCT/EP2019/054846

§ 371 Date: Aug. 26, 2020

(30) Foreign Application Priority Data
Feb. 27, 2018 (EP) 18305206.7

(51) Int. Cl. C07C 209/48; B01D 3/14; C07C 209/86

(52) U.S. Cl. CPC C07C 209/48; C07C 211/12; C07C 209/86; B01D 3/14

(57) ABSTRACT

The present invention relates to a process for separating at least one alkanolamine from a mixture comprising an aliphatic primary diamine and the at least one alkanolamine. The invention further relates to a process for the preparation of an aliphatic primary diamine by hydrogenating a dinitrile compound and separating at least one alkanolamine from the obtained mixture and to the aliphatic primary diamine obtainable by said processes.

### TECHNICAL FIELD OF THE INVENTION

The present invention relates to a process for separating at least one alkanolamine from a mixture comprising an aliphatic primary diamine and the at least one alkanolamine. The invention further relates to a process for the preparation of an aliphatic primary diamine by hydrogenating a dinitrile compound and separating at least one alkanolamine from the obtained mixture and to the aliphatic primary diamine obtainable by said processes.

### BACKGROUND

Hexamethylenediamine (HMD) is a chemical intermediate of great importance, used in particular as monomer in the manufacture of polyamides. Thus, hexamethylenediamine is used in combination with adipic acid to form an amine salt, hexamethylenediamineadipate, also known as Nylon salt. This salt is employed in the manufacture of a poly(hexamethyleneadipamide), more commonly known as PA 6,6.

Hexamethylenediamine and other aliphatic primary diamines are also important chemical intermediates, for example in the manufacture of diisocyanate compounds.

The process for the manufacture of aliphatic primary diamines, such as hexamethylenediamine, used industrially, consists of hydrogenating a dinitrile compound, such as adiponitrile, in the presence of a hydrogenation catalyst, such as a metal catalyst of Raney type, such as Raney nickel or Raney cobalt.

After the hydrogenation process, the hydrogenated products and, in particular hexamethylenediamine, are usually recovered by a sequence of distillations intended to remove water and impurities. Such impurities are generated by the decomposition of certain compounds or by reaction between the molecules present.

Major known impurities are hexamethyleneimine (HMI), diaminocyclohexane (DCH), aminocyclopentanemethylamine (AMCPA), bishexamethylentriamine (BHT) and other imines, such as tetrahydroazepine (THA) and oligomers composed of the imines and HMD, as well as water.

The majority of these impurities are a hindrance in the use of the aliphatic primary diamine, in particular hexamethylenediamine, for example as monomer in the manufacture of polyamides. This is because they can generate impurities in the polyamide obtained, causing a yellow coloring of the latter and inhomogeneities in the material, which bring about defects and breakages, in particular during the manufacture of yarns.

Numerous processes have been proposed for purifying aliphatic primary diamines and in particular hexamethylenediamine, thereby obtaining the desired diamines with a high degree of purity.

In particular, in the production of polyamides, such as PA 6,6, for the manufacture of yarns and textile fibers, it is desirable to have aliphatic primary diamines of high purity. There is therefore still a need for further improvements in the purification of such diamines.

### SUMMARY

The present inventors now found that, under certain conditions, alkanolamines, such as 1,2-aminocyclohexanol (ACHOL) are formed as impurities. The inventors furthermore found that this additional, previously not described by-product of the nitrile hydrogenation also can cause problems in the further use of the desired diamine. Like, for example, diaminocyclohexane also 1,2-aminocyclohexanol can cause in-homogeneities in polyamides manufactured from the diamine. It is therefore desirable to also remove the alkanolamine from a mixture comprising an aliphatic primary diamine and the alkanolamine.

The present inventors found that alkanolamines can be effectively separated from a mixture comprising an aliphatic primary diamine and the alkanolamine by distillation, if the bottom pressure of the distillation apparatus used is below about 35 kPa.

One aspect of the present invention therefore provides a process for separating at least one alkanolamine from a mixture comprising an aliphatic primary diamine and the at least one alkanolamine, which process comprises subjecting the mixture to distillation using a distillation apparatus, wherein the bottom pressure of the distillation apparatus is below about 35 kPa.

Another aspect of the present invention relates to a process for the preparation of an aliphatic primary diamine by hydrogenating a dinitrile compound in the presence of a hydrogenation catalyst to obtain a mixture comprising the aliphatic primary diamine and an alkanolamine, and separating the alkanolamine from the obtained mixture by the above process.

Another aspect of the present invention relates to an aliphatic primary diamine which is obtainable by the processes according to the invention.

### DETAILED DESCRIPTION

The invention relates to a process for separating at least one alkanolamine from a mixture comprising an aliphatic primary diamine and the at least one alkanolamine, which process comprises subjecting the mixture to distillation using a distillation apparatus, wherein the bottom pressure of the distillation apparatus is below about 35 kPa.

If the term “about” is used herein before a quantitative value, the present teachings also include the specific quantitative value itself, unless specifically stated otherwise. As used herein, the term “about” refers to a ±10% variation from the nominal value unless specifically stated otherwise.

In the process according to the invention a mixture comprising an aliphatic primary diamine and at least one alkanolamine is employed. In a preferred embodiment, the aliphatic primary diamine has 4 to 12 carbon atoms, preferably 4 to 8 carbon atoms. The alkanolamine can also have 4 to 12 carbon atoms, preferably 4 to 8 carbon atoms. In one embodiment, both, the aliphatic primary diamine and the alkanolamine have 4 to 12 carbon atoms, preferably 4 to 8 carbon atoms, whereby the number of carbon atoms of the aliphatic primary diamine and the alkanolamine can be the same or different. In one embodiment, the number of carbon atoms of the aliphatic primary diamine and the alkanolamine is the same.

The carbon atoms of the aliphatic primary diamine and the alkanolamine may form, for example, linear, branched or cyclic alkyl residues. In one embodiment, the carbon atoms of the aliphatic primary diamine form a linear alkyl residue. In one embodiment, the carbon atoms of the alkanolamine form a cyclic alkyl residue.

In a preferred embodiment of the present invention, the aliphatic primary diamine is hexamethylenediamine and the alkanolamine is 1,2-aminocyclohexanol, in particular 1,6-hexamethylenediamine and 1,2-aminocyclohexanol, respectively.

The mixture comprising an aliphatic primary diamine and the at least one alkanolamine may comprise one or more aliphatic primary diamines and/or one or more alkanolamines. If the mixture contains more than one aliphatic primary diamine, one of these diamines is present as predominant component of the mixture. This diamine is called “main aliphatic primary diamine” and it represents at least 50% in weight of the mixture. The term “aliphatic primary diamine” designates the sole aliphatic primary diamine of the mixture, or the main aliphatic primary diamine as defined here.

Furthermore, the mixture may comprise additional compounds, such as water and the known by-products obtained in the hydrogenation of dinitrile compounds. For example, the mixture may additionally comprise water, hexamethylenimine, diaminocyclohexane, aminocyclopentanemethylamine, bishexamethylenetriamine and/or other imines, such as tetrahydroazepine and oligomers composed of the imines and hexamethylenediamine.

The compounds other than the aliphatic primary diamine and, in particular the at least one alkanolamine being present in the mixture employed in the process of the present invention, are usually present as by-products formed during the synthesis of the aliphatic primary diamine by hydrogenation of the corresponding dinitrile compound. Therefore, the at least one alkanolamine is present in the mixture prior to distillation in only a minor amount. In one embodiment, the mixture prior to distillation contains from about 1 to about 5,000 ppm, preferably from about 10 to about 400 ppm of the at least one alkanolamine, each based on the weight of the aliphatic primary diamine. If the mixture contains more than one alkanolamine, the amounts refer to the total amounts of all alkanolamines present. If the mixture contains more than one aliphatic primary diamine, the amounts refer to the amount of the main aliphatic primary diamine present.

It was found that the process according to the invention is particularly effective in removing alkanolamines from a mixture comprising an aliphatic primary diamine and at least alkanolamine. Furthermore, as it will be demonstrated by the examples below, the process parameters can be selected such that the mixture after distillation contains a certain low amount of alkanolamines. Therefore, in one embodiment, the process of the invention provides a mixture after distillation containing less than about 100 ppm, preferably less than about 80 ppm, more preferably less than about 10 ppm, and even more preferably less than about 5 ppm of the at least one alkanolamine, each based on the weight of the aliphatic primary diamine. If the mixture after distillation contains more than one alkanolamine, the above amounts relate to the total amount of all alkanolamines. If the mixture contains more than one aliphatic primary diamine, the amounts relate to the amount of the main aliphatic primary diamine.

The process of the present invention is carried out by subjecting the mixture to distillation, wherein the bottom pressure of the distillation apparatus is below about 35 kPa. In one embodiment, the bottom pressure of the distillation apparatus is below about 28 kPa, preferably below about 20 kPa, more preferably below about 10 kPa, and most preferably below about 5 kPa.

In a further embodiment, the bottom pressure of the distillation apparatus is at least about 0.5 kPa, preferably at least about 1 kPa, and more preferably at least about 2 kPa.

The above described upper and lower limits of the bottom pressure of the distillation apparatus may be combined with each other to form preferred ranges. Thus, the bottom pressure of the distillation apparatus may be for example in the range of at least about 0.5 kPa to below about 35 kPa or, for example, in the range of at least about 1 kPa to below about 20 kPa. Any other combinations of upper and lower limits are possible as well without limitation.

The top pressure of the distillation apparatus used in the method of the present invention is not particularly limited. However, in one embodiment, the top pressure is below about 29 kPa, preferably below about 26 kPa, more preferably below about 19 kPa, even more preferably below about 9 kPa, and most preferably below about 4 kPa, such as about 2 kPa.

In one embodiment of the present invention, the bottom pressure and the top pressure of the distillation apparatus are selected such that the pressure drop between the top and the bottom of the distillation apparatus is in the range of from about 1 kPa to about 35 kPa, preferably in the range of from about 1 kPa to about 5 kPa.

In a further embodiment of the present invention, the alkanolamine is recovered from the distillation apparatus as bottom product. The quantity of aliphatic primary diamine recovered in the bottom represents less than 50% of the aliphatic primary diamine fed in the distillation apparatus, preferably less than 10%, more preferably less than 5%.

As distillation apparatus, any distillation apparatus known to the person skilled in the art may be used in the process of the present invention. A suitable distillation apparatus is, for example, a distillation column, such as a plate column, a valve tray column, a packed column and a structured packed column. Further suitable apparatus for the distillation are, for example, described in: Kirk-Othmer, Encyclopedia of Chemical Technology, 3^{rd} edition, volume 7, John Wiley and Sons, New York, 1979, pages 870-881, such as sieve plate columns, bubble cap columns or columns packed with arranged or dumped packing.

The distillation can be carried out in a single column or a plurality of columns, such as two, three or more columns. The distillation preferably is carried out in a single column, and more preferably in a sidestream takeoff column having a dividing wall (Petlyuk column).

In one embodiment, the distillation column used in the process of the present invention has a total number of theoretical plates of at least about 20. In a preferred embodiment, the total number of theoretical plates is in the range of about 20 to about 150.

In a further embodiment, the pressure drop in the distillation column is not more than 1.5 kPa, preferably not more than 1.0 kPa, more preferably not more than 0.1 kPa, each per theoretical plate of the distillation column.

The process of the present invention is particularly suitable for separating at least one alkanolamine from a mixture comprising an aliphatic primary diamine and the at least one alkanolamine, wherein said mixture is obtained during the manufacture of the aliphatic primary diamine from a dinitrile compound by hydrogenation. The present invention therefore also relates to a process for the preparation of an aliphatic primary diamine, which comprises the steps of hydrogenating a dinitrile compound in the presence of a hydrogenation catalyst to obtain a mixture comprising the aliphatic primary diamine and at least one alkanolamine, and separating the at least one alkanolamine from the obtained mixture by the above described process.

The mixture comprising the aliphatic primary diamine and at least one alkanolamine obtained by hydrogenating a dinitrile compound (or in any other way) can either be separated directly by the above-described process or, alternatively, can be subjected to other process steps, such as purification steps, prior to the separation according to the present invention. For example, it can be advantageous to precede the separation of the at least one alkanolamine by the process of the present invention by complete or partial removal of low boilers, such as water, hexamethyleneimine and aminomethylcyclopentaneamine, if present.

The partial or complete hydrogenation of the dinitrile compound, such as adiponitrile, can be carried out according to any process known in the art.

### EXAMPLES

The following examples are obtained experimentally on laboratory columns.

### Comparative Example 1

1 kg/h of HMD with a content of 4300 ppm of ACHOL is fed into a column with 30 theoretical plates. The bottom pressure of the column is controlled at 500 mbar (50 kPa). At the top of the column, main HMD is extracted with a rate of 0.970 kg/h and the mass reflux ratio is equal to 1. ACHOL content in the HMD extracted is 155 ppm.

### Example 2

Example 1 is repeated with a bottom pressure of the column controlled at 250 mbar (25 kPa). ACHOL content in the HMD extracted is 100 ppm.

### Example 3

Example 1 is repeated with a bottom pressure of the column controlled at 50 mbar (5 kPa). ACHOL content in the HMD extracted is 37 ppm.

### Example 4

Example 1 is repeated with a bottom pressure of the column controlled at 10 mbar (1 kPa). ACHOL content in the HMD extracted is 8 ppm.

1. A process for separating at least one alkanolamine from a mixture comprising an aliphatic primary diamine and the at least one alkanolamine, the process comprising:
distilling the mixture using a distillation apparatus,
wherein a bottom pressure of the distillation apparatus is below about 35 kPa.

2. The process according to claim 1, wherein the aliphatic primary diamine has 4 to 12 carbon atoms and/or the at least one alkanolamine has 4 to 12 carbon atoms.

3. The process according to claim 1, wherein the aliphatic primary diamine is hexamethylenediamine and the at least one alkanolamine is 1,2-aminocyclohexanol.

4. The process according to claim 1, wherein the mixture prior to distillation comprises from about 1 to about 5,000 ppm of the at least one alkanolamine, each based on the weight of the aliphatic primary diamine.

5. The process according to claim 1, wherein the mixture after distillation contains less than about 100 ppm of the at least one alkanolamine, each based on the weight of the aliphatic primary diamine.

6. The process according to claim 1, wherein the bottom pressure of the distillation apparatus is below about 28 kPa.

7. The process according to claim 1, wherein the bottom pressure of the distillation apparatus is at least about 0.5 kPa.

8. The process according to claim 1, wherein a top pressure of the distillation apparatus is below about 29 kPa.

9. The process according to claim 1, wherein the pressure drop between a top pressure of the distillation apparatus and the bottom pressure of the distillation apparatus is from about 1 kPa to about 35 kPa.

10. The process according to claim 1, wherein the at least one alkanolamine is recovered as bottom product.

11. The process according to claim 1, wherein the distillation apparatus is a distillation column which has a total number of theoretical plates of at least about 20.

12. The process according to claim 11, wherein the total number of theoretical plates is in the range of from about 20 to about 150.

13. The process according to claim 11, wherein a pressure drop in the distillation column is not more than 1.5 kPa per each theoretical plate of the distillation column.

14. The process according to claim 1, further comprising, before the distilling:
hydrogenating a dinitrile compound in the presence of a hydrogenation catalyst to obtain the mixture comprising the aliphatic primary diamine and the at least one alkanolamine.

15. An aliphatic primary diamine, obtained by the process of claim 1.
